(19) United States
(12) Patent Application Publication
(10) Pub. No.: US 2015/0064220 A1
(43) Pub. Date: Mar. 5, 2015

(54) METHODS FOR MODULATING CELL-MEDIATED IMMUNITY USING HUMAN MILK OLIGOSACCHARIDES

(71) Applicant: ABBOTT LABORATORIES, ABBOTT PARK, IL (US)

(72) Inventors: Debra L. Thomas, Columbus, OH (US); Rachael Buck, Gahanna, OH (US)

(73) Assignee: ABBOTT LABORATORIES, ABBOTT PARK, IL (US)

(21) Appl. No.: 14/387,871

(22) PCT Filed: Mar. 11, 2013
(86) PCT No.: PCT/US2013/030185

§ 371 Date: Sep. 25, 2014

Related U.S. Application Data
(60) Provisional application No. 61/616,230, filed on Mar. 27, 2012.

(51) Int. Cl. A61K 31/702; A23C 9/20

(52) U.S. Cl. CPC A61K 31/702; A23C 9/206
USPC 424/278.1

(57) ABSTRACT

Disclosed are methods of enhancing cell-mediated immunity in an individual using nutritional compositions including human milk oligosaccharides. The human milk oligosaccharides are sialylated human milk oligosaccharides, fucosylated human milk oligosaccharides, or a combination of both. The human milk oligosaccharides may enhance T-cell mediated responses and T-cell regulatory responses.

### CROSS REFERENCE TO RELATED APPLICATIONS

The present application hereby claims the benefit of the provisional patent application of the same title, Ser. No. 61/616,230, filed on Mar. 27, 2012, which is incorporated by reference herein in its entirety.

### FIELD OF THE DISCLOSURE

The present disclosure relates to the use of human milk oligosaccharides for modulating and enhancing cell-mediated immunity. More particularly, the present disclosure relates to methods of using sialylated human milk oligosaccharides, fucosylated human milk oligosaccharides, or a combination of both sialylated human milk oligosaccharides and fucosylated human milk oligosaccharides, to modulate and enhance cell-mediated immunity in an individual.

### BACKGROUND

The immune response is a multi-faceted series of interactions in the body among both cellular and/or non-cellular (humoral) components reacting to a perceived insult or threat to the body. In particular, cell-mediated immunity is primarily directed to microbes that survive in phagocytes and microbes that infect non-phagocytic cells (i.e., mucosal epithelial cells), or cells that display altered self-antigens, such as cancer cells. Cell-mediated immunity is most effective in removing virus-infected cells, but also participates in defending against fungi, protozoans, cancers, and intracellular bacteria.

One immune cell type involved in cell-mediated immune responses is the thymic-derived lymphocyte, or T-cell. In the context of cell-mediated immunity, T-cells provide protection by inducing apoptosis in body cells displaying epitopes of foreign antigens on their surface, such as virus-infected cells, cells with intracellular bacteria, and cancer cells displaying tumor antigens. T-cells also produce cytokines that influence the function of other cells involved in all types of immune responses, cell-mediated or humoral, innate, or adaptive. Thus, a functional cell-mediated response is a prerequisite for optimal health in an individual.

Although beneficial as noted above, uncontrolled, inappropriate, or dysregulated immune responses are implicated in chronic inflammatory diseases, such as allergies, irritable bowel syndrome, autoimmune diseases, and the like. As such, optimal health requires functional effector immune responses balanced with appropriate regulatory immune responses.

It would therefore be desirable to provide nutritional compositions that provide individual components that will enhance cell-mediated immune responses. It would be further beneficial if the enhancement in cell-mediated immune responses could be provided without creating an over-exacerbated immune response, such as uncontrolled inflammation, which can have numerous detrimental results and ultimately outweigh the beneficial aspects.

### BRIEF SUMMARY

The present disclosure is directed to methods of enhancing cell-mediated immunity in an individual, including infants, pediatrics, adults, and older adults, using human milk oligosaccharides. In some embodiments, the human milk oligosaccharide used is a sialylated human milk oligosaccharide. In other embodiments, the human milk oligosaccharide is a fucosylated human milk oligosaccharide. In still other embodiments, a combination of a sialylated and a fucosylated human milk oligosaccharide is used to enhance the cell-mediated immunity of the individual. The human milk oligosaccharides enhance T-cell mediated responses and enhance T-cell regulatory responses.

In some embodiments the present disclosure is directed to a method of enhancing cell-mediated immunity in an individual in need thereof. The method comprises administering to the individual in need thereof a nutritional composition comprising a sialylated human milk oligosaccharide in an amount sufficient to enhance T-cell mediated responses.

In other embodiments the present disclosure is directed to a method of enhancing cell-mediated immunity in an individual in need thereof. The method comprises administering to the individual in need thereof a nutritional composition comprising a fucosylated human milk oligosaccharide in an amount sufficient to enhance T-cell regulatory responses.

In other embodiments the present disclosure is directed to a method of enhancing cell-mediated immunity in an individual in need thereof. The method comprises administering to the individual in need thereof a nutritional composition comprising a sialylated human milk oligosaccharide in an amount sufficient to enhance T-cell mediated responses and a fucosylated human milk oligosaccharide in an amount sufficient to enhance T-cell regulatory responses.

In other embodiments the present disclosure is directed to a method of enhancing T-cell mediated responses in an individual in need thereof. The method comprises administering to the individual in need thereof a nutritional composition comprising a sialylated human milk oligosaccharide in an amount sufficient to enhance T-cell mediated responses.

In other embodiments the present disclosure is directed to a method of enhancing T-cell regulatory responses in an individual in need thereof. The method comprises administering to the individual in need thereof a nutritional composition comprising a fucosylated human milk oligosaccharide in an amount sufficient to enhance T-cell regulatory responses.

It has been discovered that human milk oligosaccharides affect cell-mediated immunity and that not all human milk oligosaccharides affect cell-mediated immunity in the same manner. Specifically, it has been discovered that sialylated human milk oligosaccharides affect T-cell immune responses in a manner that is different from fucosylated human milk oligosaccharides; with both affecting in a positive manner. Sialylated human milk oligosaccharides enhance T-cell mediated responses while fucosylated human milk oligosaccharides enhance T-cell regulatory responses. As such, sialylated human milk oligosaccharides promote T-cell mediated anti-viral, anti-bacterial, anti-fungal, anti-protozoan, and anti-cancer responses while fucosylated human milk oligosaccharides induce regulatory responses, which prevent or dampen chronic inflammatory disease.

Additionally, it has been unexpectedly found that a combination of a sialylated human milk oligosaccharide and a fucosylated human milk oligosaccharide demonstrate the benefits of both increased T-cell mediated function and increased regulation such that the functionalities of the sialylated human milk oligosaccharide and the fucosylated human milk oligosaccharide are not mutually exclusive. As such, it has been found that the sialylated and fucosylated human milk oligosaccharides can be used in combination to enhance balanced immune responses by preventing or dampening infectious diseases and cancer without inducing a chronic inflammatory condition. Additionally, by varying the ratios of the sialylated and fucosylated human milk oligosaccharides when used in combination, it is now possible to dampen a chronic inflammatory condition without inducing global immunosuppression or enhance a suppressed immune condition without inducing inflammation. Because the use of the human milk oligosaccharides as described herein is not antigen specific (like vaccines, for example), they may afford global enhancement of balanced cell-mediated immunity.

### BRIEF DESCRIPTION OF THE FIGURES

The accompanying drawings, which are incorporated in and constitute a part of this specification, illustrate embodiments, and together with the general description given above, and the detailed description of the embodiments given below, serve to explain the principles of the present disclosure.

FIG. 1 is a graph depicting PHA-stimulated responses of PBMCs in the presence of individual HMOs and HMO mixtures as analyzed in Example 11.

FIG. 2 is a graph depicting proliferative responses of PBMCs in the presence of individual HMOs and HMO mixtures as analyzed in Example 11.

FIG. 3 is a graph depicting the percentage of T-regulatory cells in mesenteric lymph nodes of mice supplemented with and without 2′FL as analyzed in Example 12.

### DETAILED DESCRIPTION

The present disclosure is directed to methods for providing a global enhancement of cell-mediated immunity in an individual. Unlike many other conventional methods that utilize one or more specific nutritional components or synthetic drugs to enhance cell-mediated immunity in an individual through either enhanced T-cell mediated responses or enhanced T-cell regulatory responses, the methods of the present disclosure provide enhancement of cell-mediated immunity by simultaneously enhancing both T-cell mediated responses and enhancing T-cell regulatory responses. This allows for enhanced immunological benefits for an individual without the increased risk of unwanted inflammation.

These dual benefits are obtained in the methods of the present disclosure by using a combination of human milk oligosaccharides in a nutritional composition. Because it has been unexpectedly found that the dual benefits are not mutually exclusive and can be provided in a single nutritional composition, the methods of the present disclosure are particularly beneficial. In some embodiments, the nutritional compositions include a first sialylated human milk oligosaccharide and a second fucosylated human milk oligosaccharide. Depending upon the desired outcome for the individual, the amounts and ratios of the two human milk oligosaccharides can be varied to produce the desired outcome.

These and other features of the nutritional compositions and methods, as well as some of the many optional variations and additions, are described in detail hereafter.

The terms “cell-mediated immunity” and “T-cell mediated immunity” as used herein, unless otherwise specified, refers to the immune response produced when naïve or sensitized T-cells directly attack foreign antigens and secrete cytokines that initiate the body's humoral immune response.

The terms “retort packaging” and “retort sterilizing” are used interchangeably herein, and unless otherwise specified, refer to the common practice of filling a container, most typically a metal can or other similar package, with a nutritional liquid and then subjecting the liquid-filled package to the necessary heat sterilization step, to form a sterilized, retort packaged, nutritional liquid product.

The term “aseptic packaging” as used herein, unless otherwise specified, refers to the manufacture of a packaged product without reliance upon the above-described retort packaging step, wherein the nutritional liquid and package are sterilized separately prior to filling, and then are combined under sterilized or aseptic processing conditions to form a sterilized, aseptically packaged, nutritional liquid product.

The terms “fat” and “oil” as used herein, unless otherwise specified, are used interchangeably to refer to lipid materials derived or processed from plants or animals. These terms also include synthetic lipid materials so long as such synthetic materials are suitable for oral administration to humans.

The term “human milk oligosaccharide” or “HMO”, as used herein, unless otherwise specified, refers generally to a number of complex carbohydrates found in human breast milk that can be in acidic or neutral form, and to precursors thereof. Exemplary non-limiting human milk oligosaccharides include 3′-sialyllactose, 6′-sialyllactose, 3′-fucosyllactose, 2′-fucosyllactose, and lacto-N-neo-tetraose. Exemplary human milk oligosaccharide precursors include sialic acid and/or fucose.

The term “shelf stable” as used herein, unless otherwise specified, refers to a nutritional product that remains commercially stable after being packaged and then stored at 18-24° C. for at least 3 months, including from about 6 months to about 24 months, and also including from about 12 months to about 18 months.

The terms “nutritional formulation” or “nutritional composition” as used herein, are used interchangeably and, unless otherwise specified, refer to synthetic formulas including nutritional liquids, nutritional powders, nutritional solids, nutritional semi-solids, nutritional semi-liquids, nutritional supplements, and any other nutritional food product as known in the art. The nutritional powders may be reconstituted to form a nutritional liquid, all of which comprise one or more of fat, protein and carbohydrate and are suitable for oral consumption by a human.

The term “nutritional liquid” as used herein, unless otherwise specified, refers to nutritional products in ready-to-drink liquid form, concentrated form, and nutritional liquids made by reconstituting the nutritional powders described herein prior to use.

The term “nutritional powder” as used herein, unless otherwise specified, refers to nutritional products in flowable or scoopable form that can be reconstituted with water or another aqueous liquid prior to consumption and includes both spray dried and drymixed/dryblended powders.

The term “nutritional semi-solid,” as used herein, unless otherwise specified, refers to nutritional products that are intermediate in properties, such as rigidity, between solids and liquids. Some semi-solids examples include puddings, gelatins, and doughs.

The term “nutritional semi-liquid,” as used herein, unless otherwise specified, refers to nutritional products that are intermediate in properties, such as flow properties, between liquids and solids. Some semi-liquids examples include thick shakes and liquid gels.

The terms “susceptible” and “at risk” as used herein, unless otherwise specified, mean having little resistance to a certain condition or disease, including being genetically predisposed, having a family history of, and/or having symptoms of the condition or disease.

The terms “modulating” or “modulation” or “modulate” as used herein, unless otherwise specified, refer to the targeted movement of a selected characteristic.

### Product Form

The nutritional compositions used in the methods of the present disclosure include a sialylated human milk oligosaccharide, a fucosylated human milk oligosaccharide, or a combination of a sialylated and a fucosylated human milk oligosaccharide and may be formulated and administered in any known or otherwise suitable oral product form. Any solid, liquid, semi-solid, semi-liquid, or powder product form, including combinations or variations thereof, are suitable for use herein, provided that such forms allow for safe and effective oral delivery to the individual of the ingredients as also defined herein.

The nutritional compositions used in the methods of the present disclosure are desirably formulated as dietary product forms, which are defined herein as those embodiments comprising the ingredients of the present disclosure in a product form that then contains at least one of fat, protein, and carbohydrate, and preferably also contains vitamins, minerals, or combinations thereof.

The nutritional compositions may be formulated with sufficient kinds and amounts of nutrients to provide a sole, primary, or supplemental source of nutrition, or to provide a specialized nutritional product for use in individuals afflicted with specific diseases or conditions or with a targeted nutritional benefit as described below.

Some exemplary, non-limiting, examples of specific products that may be suitable for use in accordance with the present disclosure include preterm infant formulas, term infant formulas, human milk fortifiers, pediatric formulas, adult nutritional formulas, older adult nutritional formulas, medical formulas, geriatric nutritional formulas, diabetic nutritional formulas, and the like.

### Nutritional Liquids

Nutritional liquids include both concentrated and ready-to-feed nutritional liquids. These nutritional liquids are most typically formulated as suspensions or emulsions, although other liquid forms are within the scope of the present disclosure.

Nutritional emulsions suitable for use may be aqueous emulsions comprising proteins, fats, and carbohydrates. These emulsions are generally flowable or drinkable liquids at from about 1° C. to about 25° C. and are typically in the form of oil-in-water, water-in-oil, or complex aqueous emulsions, although such emulsions are most typically in the form of oil-in-water emulsions having a continuous aqueous phase and a discontinuous oil phase.

The nutritional emulsions may be and typically are shelf stable. The nutritional emulsions typically contain up to 95% by weight of water, including from about 50% to 95%, also including from about 60% to about 90%, and also including from about 70% to about 85%, of water by weight of the nutritional emulsions. The nutritional emulsions may have a variety of product densities, but most typically have a density greater than 1.03 g/mL, including greater than 1.04 g/mL, including greater than 1.055 g/mL, including from about 1.06 g/mL to about 1.12 g/mL, and also including from about 1.085 g/mL to about 1.10 g/mL.

The nutritional emulsions may have a caloric density tailored to the nutritional needs of the ultimate user, although in most instances the emulsions comprise generally at least 19 kcal/fl oz (660 kcal/liter), more typically from about 20 kcal/fl oz (675-680 kcal/liter) to about 25 kcal/fl oz (820 kcal/liter), even more typically from about 20 kcal/fl oz (675-680 kcal/liter) to about 24 kcal/fl oz (800-810 kcal/liter). Generally, the 22-24 kcal/fl oz formulas are more commonly used in preterm or low birth weight infants, and the 20-21 kcal/fl oz (675-680 to 700 kcal/liter) formulas are more often used in term infants. In some embodiments, the emulsion may have a caloric density of from about 50-100 kcal/liter to about 660 kcal/liter, including from about 150 kcal/liter to about 500 kcal/liter. In some specific embodiments, the emulsion may have a caloric density of 25, or 50, or 75, or 100 kcal/liter.

The nutritional emulsion may have a pH ranging from about 3.5 to about 8, but are most advantageously in a range of from about 4.5 to about 7.5, including from about 5.5 to about 7.3, including from about 6.2 to about 7.2.

Although the serving size for the nutritional emulsion can vary depending upon a number of variables, a typical serving size is generally at least 1 mL, or even at least 2 mL, or even at least 5 mL, or even at least 10 mL, or even at least 25 mL, including ranges from 1 mL to about 300 mL, including from about 4 mL to about 250 mL, and including from about 10 mL to about 240 mL.

### Nutritional Solids

The nutritional solids may be in any solid form but are typically in the form of flowable or substantially flowable particulate compositions, or at least particulate compositions. Particularly suitable nutritional solid product forms include spray dried, agglomerated and/or dryblended powder compositions. The compositions can easily be scooped and measured with a spoon or similar other device, and can easily be reconstituted by the intended user with a suitable aqueous liquid, typically water, to form a nutritional composition for immediate oral or enteral use. In this context, “immediate” use generally means within about 48 hours, most typically within about 24 hours, preferably right after reconstitution.

The nutritional powders may be reconstituted with water prior to use to a caloric density tailored to the nutritional needs of the ultimate user, although in most instances the powders are reconstituted with water to form compositions comprising at least 19 kcal/fl oz (660 kcal/liter), more typically from about 20 kcal/fl oz (675-680 kcal/liter) to about 25 kcal/fl oz (820 kcal/liter), even more typically from about 20 kcal/fl oz (675-680 kcal/liter) to about 24 kcal/fl oz (800-810 kcal/liter). Generally, the 22-24 kcal/fl oz formulas are more commonly used in preterm or low birth weight infants, and the 20-21 kcal/fl oz (675-680 to 700 kcal/liter) formulas are more often used in term infants. In some embodiments, the reconstituted powder may have a caloric density of from about 50-100 kcal/liter to about 660 kcal/liter, including from about 150 kcal/liter to about 500 kcal/liter. In some specific embodiments, the emulsion may have a caloric density of 25, or 50, or 75, or 100 kcal/liter.

### Methods Of Enhancing Cell-Mediated Immunity

The methods of the present disclosure use sialylated human milk oligosaccharides, fucosylated human milk oligosaccharides, or a combination thereof to modulate and/or enhance the cell-mediated immunity responses of an individual. As noted, cell-mediated immunity is directed primarily at microbes that survive in phagocytes and microbes that infect non-phagocytic cells (i.e., mucosal epithelial cells) or cells that display altered self-antigens, such as cancer cells. Cell-mediated immunity is most effective in removing virus-infected cells, but also defends against fungi, protozoans, cancers, and intracellular bacteria. The methods of the present disclosure that modulate and/or enhance cell mediated immunity are beneficial for a wide range of individuals, including preterm infants, infants, pediatric individuals, teens, adults, and older adults (adults at least 50 or more years of age).

In some embodiments of the present disclosure, cell-mediated immunity is enhanced in an individual by administering to the individual an effective amount of a sialylated human milk oligosaccharide that enhances the T-cell mediated responses in the individual. Upon administration to an individual, the sialylated human milk oligosaccharides promote T-cell mediated anti-viral, anti-bacterial, anti-fungal, anti-protozoan, and anti-cancer responses in the individual to enhance the cell-mediated responses in the individual.

The sialylated human milk oligosaccharide may be administered to generally healthy individuals, or may be administered to a subset of individuals in need of enhanced T-cell mediated responses. Some individuals that are in specific need of enhanced T-cell mediated responses may include allergy prone infants, pediatrics, teens, or adults (infants, pediatrics, teens, or adults susceptible to or at elevated risk of allergies), autoimmune disease prone infants, pediatrics, teens or adults (infants, pediatrics, teens or adults susceptible to or at elevated risk of autoimmune disease), non-breastfed infants, breastfed infants supplemented with formula or human milk fortifier, immunosenescent adults and older adults, menopausal women, post menopausal women, post-chemotherapy patients, individuals with compromised immune systems, individuals with AIDS, and the like. Preterm infants, infants, pediatrics, teens, adults, and older adults may be susceptible to or at elevated risk for a condition or disease due to family history, age, environment, and/or lifestyle. Based on the foregoing, because some of the method embodiments of the present disclosure utilizing the sialylated human milk oligosaccharides are directed to specific subsets or subclasses of identified individuals (that is, the subset or subclass of individuals “in need” of assistance in addressing one or more specific diseases or specific conditions noted herein), not all individuals will fall within the subset or subclass of individuals as described herein for certain diseases or conditions.

In other embodiments of the present disclosure, cell-mediated immunity is enhanced in an individual by administering to the individual an effective amount of a fucosylated human milk oligosaccharide that enhances the T-cell regulatory responses in the individual. Upon administration to an individual, the fucosylated human milk oligosaccharides induce regulatory responses that prevent or dampen chronic disease or inflammation to enhance the T-cell regulatory responses in the individual.

The fucosylated human milk oligosaccharide may be administered to generally healthy individuals, or may be administered to a subset of individuals in need of enhanced T-cell regulatory responses. Some individuals that are in specific need of enhanced T-cell regulatory responses may include allergy prone infants, pediatrics, teens or adults (infants, pediatrics, teens or adults susceptible to or at elevated risk of allergies), autoimmune disease prone infants, pediatrics, teens or adults (infants, pediatrics, teens or adults susceptible to or at elevated risk of autoimmune disease), non-breastfed infants, breastfed infants supplemented with formula or human milk fortifier, older adults, immunosenescent adults and older adults, menopausal women, post menopausal women, individuals with chronic inflammatory bowel disease, and the like. Preterm infants, infants, pediatrics, teens adults, and older adults may be susceptible to or at elevated risk for a condition or disease due to family history, age, environment, and/or lifestyle. Based on the foregoing, because some of the method embodiments of the present disclosure using the fucosylated human milk oligosaccharides are directed to specific subsets or subclasses of identified individuals (that is, the subset or subclass of individuals “in need” of assistance in addressing one or more specific diseases or specific conditions noted herein), not all individuals will fall within the subset or subclass of individuals as described herein for certain diseases or conditions.

In another desirable embodiment of the present disclosure, cell-mediated immunity is enhanced in an individual by administering to the individual an effective amount of a combination of a sialylated human milk oligosaccharide that enhances the T-cell mediated responses in the individual and a fucosylated human milk oligosaccharide that induces regulatory responses in the individual. Upon administration to an individual, the sialylated human milk oligosaccharides promote T-cell mediated anti-viral, anti-bacterial, anti-fungal, anti-protozoan, and anti-cancer responses in the individual to enhance the cell-mediated responses in the individual, and the fucosylated human milk oligosaccharides induce regulatory responses that prevent or dampen chronic disease or inflammation. Unexpectedly, a combination of sialylated and fucosylated human milk oligosaccharides demonstrates the benefits of both increased T-cell mediated responses/function and increased regulation. As such, it has been surprisingly found that the functionalities of the sialylated and fucosylated human milk oligosaccharides are not mutually exclusive and they can be used in combination to enhance balanced immune responses in an individual by preventing or dampening infectious diseases and cancer without inducing a chronic inflammatory response or condition that can reduce the overall benefit from the administration of the human milk oligosaccharides. Additionally, by varying the ratios of amounts of sialylated and fucosylated human milk oligosaccharides in the nutritional composition administered to the individual, it is now possible to suppress a chronic inflammatory condition without inducing global immunosuppression or enhance a suppressed immune condition without inducing unwanted inflammation.

The combination of sialylated and fucosylated human milk oligosaccharides may be administered to generally healthy individuals, or may be administered to a subset of individuals in need of enhanced T-cell mediated responses and T-cell regulatory responses. Some individuals that are in specific need of enhanced T-cell mediated responses and enhanced T-cell regulatory responses may include allergy prone infants, pediatrics, teens, or adults (infants, pediatrics, teens, or adults susceptible to or at elevated risk of allergies), autoimmune disease prone infants, pediatrics, teens, or adults (infants, pediatrics, teens, or adults susceptible to or at elevated risk of autoimmune disease), non-breastfed infants, breastfed infants supplemented with formula or human milk fortifier, older adults, immunosenescent adults and older adults, menopausal women, post menopausal women, post-chemotherapy patients, individuals with compromised immune systems, individuals with AIDS, individuals with chronic inflammatory bowel disease, and the like. Preterm infants, infants, pediatrics, teens, adults, and older adults may be susceptible to or at elevated risk for a condition or disease due to family history, age, environment, and/or lifestyle. Based on the foregoing, because some of the method embodiments of the present disclosure using the sialylated and fucosylated human milk oligosaccharides are directed to specific subsets or subclasses of identified individuals (that is, the subset or subclass of individuals “in need” of assistance in addressing one or more specific diseases or specific conditions noted herein), not all individuals will fall within the subset or subclass of individuals as described herein for certain diseases or conditions.

In one example of the present disclosure, a preterm or term infant formula may include a combination of both sialylated and fucosylated human milk oligosaccharides to support balanced effector immune responses. In another example of the present disclosure, a preterm or term infant formula may include higher levels of fucosylated human milk oligosaccharides as compared to sialylated human milk oligosaccharides to prevent or reduce allergic diseases, autoimmune diseases, or chronic inflammatory diseases. In another example of the present disclosure, an adult nutritional may include higher levels of fucosylated human milk oligosaccharides as compared to sialylated human milk oligosaccharides to dampen allergic diseases, autoimmune diseases or chronic inflammatory diseases. In another example of the present disclosure, an adult nutritional may contain higher levels of sialylated human milk oligosaccharides as compared to fucosylated human milk oligosaccharides and be taken in combination with cancer therapies or therapies directed at HIV and AIDS.

### Sialylated and Fucosylated Human Milk Oligosaccharides

The methods of the present disclosure for modulating cell-mediated immunity utilize nutritional compositions that include a sialylated human milk oligosaccharide, a fucosylated human milk oligosaccharide, or a combination of a sialylated human milk oligosaccharide and a fucosylated human milk oligosaccharide. In some embodiments, the nutritional compositions include a sialylated human milk oligosaccharide and are substantially free of a fucosylated human milk oligosaccharide. In other embodiments, the nutritional compositions include a fucosylated human milk oligosaccharide and are substantially free of a sialylated human milk oligosaccharide.

The nutritional compositions may optionally include one or more additional human milk oligosaccharides as noted herein. The human milk oligosaccharide(s) used in the nutritional composition may be isolated or enriched from milk(s) secreted by mammals including, but not limited to: human, bovine, ovine, porcine, or caprine species. The human milk oligosaccharides may also be produced via microbial fermentation, enzymatic processes, chemical synthesis, or combinations thereof.

Suitable sialylated human milk oligosaccharides for use in the nutritional compositions include at least one sialic acid residue in the oligosaccharide backbone. The sialylated human milk oligosaccharide may include two or more sialic acid residues also. Specific non-limiting examples of sialylated human milk oligosaccharides for use in the present disclosure include sialyl oligosaccharides, sialic acid (i.e., free sialic acid, lipid-bound sialic acid, protein-bound sialic acid), lactosialotetraose, 3′-Sialyl-3-fucosyllactose, Disialomonofucosyllacto-N-neohexaose, Monofucosylmonosialyllacto-N-octaose (sialyl Lea), Sialyllacto-N-fucohexaose II, Disialyllacto-N-fucopentaose II, Monofucosyldisialyllacto-N-tetraose), sialyl fucosyl oligosaccharides, 2′-Sialyllactose, 2-Sialyllactosamine, 3′-Sialyllactose, 3′-Sialyllactosamine, 6′-Sialyllactose, 6′-Sialyllactosamine, Sialyllacto-N-neotetraose c, Monosialyllacto-N-hexaose, Disialyllacto-N-hexaose I, Monosialyllacto-N-neohexaose I, Monosialyllacto-N-neohexaose II, Disialyllacto-N-neohexaose, Disialyllacto-N-tetraose, Disialyllacto-N-hexaose II, Sialyllacto-N-tetraose a, Disialyllacto-N-hexaose I, Sialyllacto-N-tetraose b, sialyl-lacto-N-tetraose a, sialyl-lacto-N-tetraose b, sialyl-lacto-N-tetraose c, sialyl-fucosyl-lacto-N-tetraose I, sialyl-fucosyl-lacto-N-tetraose II, disialyl-lacto-N-tetraose and combinations thereof. Particularly desirable sialylated human milk oligosaccharides include 3′Sialyllactose, 6′Sialyllactose, and combinations thereof.

Specific non-limiting examples of fucosylated human milk oligosaccharides for use in the present disclosure include fucosyl oligosaccharides, Lacto-N-fucopentaose I, Lacto-N-fucopentaose II, 2′-Fucosyllactose, 3′-Fucosyllactose, Lacto-N-fucopentaose III, Lacto-N-difucohexaose I, Lactodifucotetraose, monofucosyllacto-N-hexaose II, isomeric fucosylated lacto-N-hexaose (1), isomeric fucosylated lacto-N-hexaose (3), isomeric fucosylated lacto-N-hexaose (2), difucosyl-para-lacto-N-neohexaose, difucosyl-para-lacto-N-hexaose, difucosyllacto-N-hexaosemonofucosyllacto-neoocataose, monofucosyllacto-N-ocataose, difucosyllacto-N-octaose I, difucosyllacto-N-octaose II, difucosyllacto-N-neoocataose II, difucosyllacto-N-neoocataose I, lacto-N-fucopentaose V, lacto-N-decaose, trifucosyllacto-N-neooctaose, trifucosyllacto-N-octaose, trifucosyl-iso-lacto-N-octaose, lacto-N-difuco-hexaose II, and combinations thereof. Particularly desirable fucosylated human milk oligosaccharides include 2′-Fucosyllactose, 3′-Fucosyllactose, and combinations thereof.

Other suitable examples of human milk oligosaccharides that may be included in the nutritional compositions for use in the methods of the present disclosure include lacto-N-hexaose, para-lacto-N-hexaose, lacto-N-neohexaose, para-lacto-N-neohexaose, lacto-N-neoocataose, para-lacto-N-octanose, iso-lacto-N-octaose, lacto-N-octaose, and combinations thereof.

The sialylated and fucosylated human milk oligosaccharides (and any additional optional human milk oligosaccharides) are present in the nutritional compositions in a total amount of human milk oligosaccharide in the nutritional composition (mg of human milk oligosaccharide per mL of composition) of at least 0.001 mg/mL, including at least 0.01 mg/mL, including from 0.001 mg/mL to about 20 mg/mL, including from about 0.01 mg/mL to about 20 mg/mL, including from 0.001 mg/mL to about 10 mg/mL, including from about 0.01 mg/mL to about 10 mg/mL, including from 0.001 mg/mL to about 5 mg/mL, including from about 0.01 mg/mL to about 5 mg/mL, including from 0.001 mg/mL to about 1 mg/mL, including from 0.001 mg/mL to about 0.23 mg/mL, including from about 0.01 mg/mL to about 0.23 mg/mL of total human milk oligosaccharide in the nutritional composition. Typically, the amount of specific sialylated human milk oligosaccharide and/or fucosylated human milk oligosaccharide present in the nutritional composition will depend on the specific human milk oligosaccharide or human milk oligosaccharides present and the amounts of other components in the nutritional compositions, including the amounts any optional human milk oligosaccharides.

In one embodiment when the nutritional composition is a nutritional powder, the total concentration of human milk oligosaccharide in the nutritional powder is from about 0.0005% to about 5%, including from about 0.01% to about 1% (by weight of the nutritional powder).

In other embodiments, when the nutritional product is a ready-to-feed nutritional liquid, the total concentration of human milk oligosaccharides in the ready-to-feed nutritional liquid is from about 0.0001% to about 0.50%, including from about 0.001% to about 0.15%, including from about 0.01% to about 0.10%, and further including from about 0.01% to about 0.03% (by weight of the ready-to-feed nutritional liquid).

In other embodiments when the nutritional product is a concentrated nutritional liquid, the total concentration of human milk oligosaccharides in the concentrated nutritional liquid is from about 0.0002% to about 0.60%, including from about 0.002% to about 0.30%, including from about 0.02% to about 0.20%, and further including from about 0.02% to about 0.06% (by weight of the concentrated nutritional liquid).

### Long Chain Polyunsaturated Fatty Acids (LCPUFAs)

In addition to the human milk oligosaccharides described above, the nutritional compositions may optionally include LCPUFAs. LCPUFAs are included in the nutritional compositions to provide nutritional support, as well as to reduce oxidative stress and enhance growth and functional development of the intestinal epithelium and associated immune cell populations.

Exemplary LCPUFAs for use in the nutritional compositions include, for example, ω-3 LCPUFAs and ω-6 LCPUFAs. Specific LCPUFAs include docosahexaenoic acid (DHA), eicosapentaenoic acid (EPA), docosapentaenoic acid (DPA), arachidonic acid (ARA), linoleic acid, linolenic acid (alpha linolenic acid) and gamma-linolenic acid derived from oil sources such as plant oils, marine plankton, fungal oils, and fish oils. In one particular embodiment, the LCPUFAs are derived from fish oils such as menhaden, salmon, anchovy, cod, halibut, tuna, or herring oil. Particularly preferred LCPUFAs for use in the nutritional compositions with the HMOs include DHA, ARA, EPA, DPA, and combinations thereof.

In order to reduce potential side effects of high dosages of LCPUFAs in the nutritional compositions, the content of LCPUFAs preferably does not exceed about 3% by weight of the total fat content, including below 2% by weight of the total fat content, and including below 1% by weight of the total fat content in the nutritional composition.

The LCPUFA may be provided as free fatty acids, in triglyceride form, in diglyceride form, in monoglyceride form, in phospholipid form, in esterified form or as a mixture of one or more of the above, preferably in triglyceride form.

The nutritional compositions as described herein will typically comprise total concentrations of LCPUFA of from about 0.01 mM to about 10 mM and including from about 0.01 mM to about 1 mM. Alternatively, the nutritional compositions comprise total concentrations of LCPUFA of from about 0.001 g/L to about 1 g/L.

### Antioxidants

Additionally, the nutritional compositions may optionally include one or more antioxidants in combination with the human milk oligosaccharides to provide nutritional support, as well as to reduce oxidative stress.

Any antioxidants suitable for oral administration may be included for use in the nutritional compositions of the present disclosure, including, for example, vitamin A, vitamin E, vitamin C, retinol, tocopherol, and carotenoids, including lutein, beta-carotene, zeaxanthin, and lycopene, and combinations thereof, for example.

The nutritional compositions may comprise at least one of lutein, lycopene, zeaxanthin, and beta-carotene to provide a total amount of carotenoid of from about 0.001 μg/mL to about 10 μg/mL. More particularly, the nutritional compositions comprise lutein in an amount of from about 0.001 μg/mL to about 10 μg/mL, including from about 0.001 μg/mL to about 5 μg/mL, including from about 0.001 μg/mL to about 0.0190 μg/mL, including from about 0.001 μg/mL to about 0.0140 μg/mL, and also including from about 0.044 μg/mL to about 5 μg/mL of lutein. It is also generally preferable that the nutritional compositions comprise from about 0.001 μg/mL to about 10 μg/mL, including from about 0.001 μg/mL to about 5 μg/mL, from about 0.001 μg/mL to about 0.0130 μg/mL, including from about 0.001 μg/mL to about 0.0075 μg/mL, and also including from about 0.0185 μg/mL to about 5 μg/mL of lycopene. It is also generally preferable that the nutritional compositions comprise from about 1 μg/mL to about 10 μg/mL, including from about 1 μg/mL to about 5 μg/mL, including from about 0.001 μg/mL to about 0.025 μg/mL, including from about 0.001 μg/mL to about 0.011 μg/mL, and also including from about 0.034 μg/mL to about 5 μg/mL of beta-carotene. It should be understood that any combination of these amounts of beta-carotene, lutein, zeaxanthin, and lycopene can be included in the nutritional compositions of the present disclosure. Other carotenoids may optionally be included in the nutritional compositions as described herein. Any one or all of the carotenoids included in the nutritional compositions described herein may be from a natural source, or artificially synthesized.

### Nucleotides

In addition to the human milk oligosaccharides, the nutritional compositions of the present disclosure may additionally optionally include nucleotides and/or nucleotide precursors selected from the group consisting of nucleosides, purine bases, pyrimidine bases, ribose and deoxyribose. The nucleotide may be in monophosphate, diphosphate, or triphosphate form. The nucleotide may be a ribonucleotide or a deoxyribonucleotide. The nucleotides may be monomeric, dimeric, or polymeric (including RNA and DNA). The nucleotide may be present in the nutritional composition as a free acid or in the form of a salt, preferably a monosodium salt.

Suitable nucleotides and/or nucleosides for use in the nutritional compositions include one or more of cytidine 5′-monophosphate, uridine 5′-monophosphate, adenosine 5′-monophosphate, guanosine 5′-1-monophosphate, and/or inosine 5′-monophosphate, more preferably cytidine 5′-monophosphate, uridine 5′-monophosphate, adenosine 5′-monophosphate, guanosine 5′-monophosphate, and inosine 5′-monophosphate.

The nucleotides are present in the nutritional products in total amounts of nucleotides of at least 5 mg/L, including at least 10 mg/L, including from about 10 mg/L to about 200 mg/L, including from about 42 mg/L to about 102 mg/L, and including at least about 72 mg/L of the nutritional product.

### Macronutrients

The nutritional compositions including the human milk oligosaccharide may be formulated to include at least one of protein, fat, and carbohydrate. In many embodiments, the nutritional compositions will include the human milk oligosaccharide with protein, carbohydrate and fat.

Although total concentrations or amounts of the fat, protein, and carbohydrates may vary depending upon the product type (i.e., human milk fortifier, preterm infant formula, infant formula, pediatric formula, adult formula, medical formula, etc.), product form (i.e., nutritional solid, powder, ready-to-feed liquid, or concentrated liquid) and targeted dietary needs of the intended user, such concentrations or amounts most typically fall within one of the following embodied ranges, inclusive of any other essential fat, protein, and/or carbohydrate ingredients as described herein.

For infant and adult formulas, carbohydrate concentrations most typically range from about 5% to about 40%, including from about 7% to about 30%, including from about 10% to about 25%, by weight; fat concentrations most typically range from about 1% to about 30%, including from about 2% to about 15%, and also including from about 3% to about 10%, by weight; and protein concentrations most typically range from about 0.5% to about 30%, including from about 1% to about 15%, and also including from about 2% to about 10%, by weight.

The amount of carbohydrates, fats, and/or proteins in any of the liquid nutritional compositions described herein may also be characterized in addition to, or in the alternative, as a percentage of total calories in the liquid nutritional composition as set forth in the following table. These macronutrients for liquid nutritional compositions of the present disclosure are most typically formulated within any of the caloric ranges (embodiments A-F) described in the following table (each numerical value is preceded by the term “about”).

| Nutrient % Total Cal. | Embodiment A | Embodiment B | Embodiment C |
|---|---|---|---|
| Carbohydrate | 0-98 | 2-96 | 10-75 |
| Protein | 0-98 | 2-96 | 5-70 |
| Fat | 0-98 | 2-96 | 20-85 |

| Nutrient % Total Cal. | Embodiment D | Embodiment E | Embodiment F |
|---|---|---|---|
| Carbohydrate | 30-50 | 25-50 | 25-50 |
| Protein | 15-35 | 10-30 | 5-30 |
| Fat | 35-55 | 1-20 | 2-20 |

In one specific example, liquid infant formulas (both ready-to-feed and concentrated liquids) include those embodiments in which the protein component may comprise from about 7.5% to about 25% of the caloric content of the formula; the carbohydrate component may comprise from about 35% to about 50% of the total caloric content of the infant formula; and the fat component may comprise from about 30% to about 60% of the total caloric content of the infant formula. These ranges are provided as examples only, and are not intended to be limiting. Additional suitable ranges are noted in the following table (each numerical value is preceded by the term “about”).

| Nutrient % Total Cal. | Embodiment G | Embodiment H | Embodiment I |
|---|---|---|---|
| Carbohydrates: | 20-85 | 30-60 | 35-55 |
| Fat: | 5-70 | 20-60 | 25-50 |
| Protein: | 2-75 | 5-50 | 7-40 |

When the nutritional product is a powdered preterm or term infant formula, the protein component is present in an amount of from about 5% to about 35%, including from about 8% to about 12%, and including from about 10% to about 12% by weight of the preterm or term infant formula; the fat component is present in an amount of from about 10% to about 35%, including from about 25% to about 30%, and including from about 26% to about 28% by weight of the preterm or term infant formula; and the carbohydrate component is present in an amount of from about 30% to about 85%, including from about 45% to about 60%, including from about 50% to about 55% by weight of the preterm or term infant formula.

For powdered human milk fortifiers the protein component is present in an amount of from about 1% to about 55%, including from about 10% to about 50%, and including from about 10% to about 30% by weight of the human milk fortifier; the fat component is present in an amount of from about 1% to about 30%, including from about 1% to about 25%, and including from about 1% to about 20% by weight of the human milk fortifier; and the carbohydrate component is present in an amount of from about 15% to about 75%, including from about 15% to about 60%, including from about 20% to about 50% by weight of the human milk fortifier.

The total amount or concentration of fat, carbohydrate, and protein, in the powdered nutritional compositions of the present disclosure can vary considerably depending upon the selected composition and dietary or medical needs of the intended user. Additional suitable examples of macronutrient concentrations are set forth below. In this context, the total amount or concentration refers to all fat, carbohydrate, and protein sources in the powdered product. For powdered nutritional compositions, such total amounts or concentrations are most typically and preferably formulated within any of the embodied ranges described in the following table (each numerical value is preceded by the term “about”).

| Nutrient % Total Cal. | Embodiment J | Embodiment K | Embodiment L |
|---|---|---|---|
| Carbohydrate | 1-85 | 30-60 | 35-55 |
| Fat | 5-70 | 20-60 | 25-50 |
| Protein | 2-75 | 5-50 | 7-40 |

### Fat

The nutritional compositions used in the methods of the present disclosure may include a source or sources of fat. Suitable additional sources of fat for use herein include any fat or fat source that is suitable for use in an oral nutritional product and is compatible with the elements and features of such products. For example, in one specific embodiment, the additional fat is derived from short chain fatty acids.

Additional non-limiting examples of suitable fats or sources thereof for use in the nutritional products described herein include coconut oil, fractionated coconut oil, soybean oil, corn oil, olive oil, safflower oil, high oleic safflower oil, oleic acids (EMERSOL 6313 OLEIC ACID, Cognis Oleochemicals, Malaysia), MCT oil (medium chain triglycerides), sunflower oil, high oleic sunflower oil, palm and palm kernel oils, palm olein, canola oil, marine oils, fish oils, fungal oils, algae oils, cottonseed oils, and combinations thereof

### Protein

The nutritional compositions used in the methods of the present disclosure may optionally further comprise protein. Any protein source that is suitable for use in oral nutritional compositions and is compatible with the elements and features of such products is suitable for use in the nutritional compositions.

Non-limiting examples of suitable proteins or sources thereof for use in the nutritional products include hydrolyzed, partially hydrolyzed or non-hydrolyzed proteins or protein sources, which may be derived from any known or otherwise suitable source such as milk (e.g., casein, whey), animal (e.g., meat, fish), cereal (e.g., rice, corn), vegetable (e.g., soy, pea) or combinations thereof. Non-limiting examples of such proteins include milk protein isolates, milk protein concentrates as described herein, casein protein isolates, extensively hydrolyzed casein, whey protein, sodium or calcium caseinates, whole cow milk, partially or completely defatted milk, soy protein isolates, soy protein concentrates, intact pea protein concentrates, intact pea protein isolates, hydrolyzed pea protein concentrates, hydrolyzed pea protein isolates, and so forth. In one specific embodiment, the nutritional compositions include a protein source derived from milk proteins of human and/or bovine origin.

### Carbohydrate

The nutritional products as used in the methods of the present disclosure may further optionally comprise any carbohydrates that are suitable for use in an oral nutritional product and are compatible with the elements and features of such products.

Non-limiting examples of suitable carbohydrates or sources thereof for use in the nutritional products described herein may include maltodextrin, hydrolyzed or modified starch or cornstarch, glucose polymers, corn syrup, corn syrup solids, rice-derived carbohydrates, pea-derived carbohydrates, potato-derived carbohydrates, tapioca, sucrose, glucose, fructose, lactose, high fructose corn syrup, honey, sugar alcohols (e.g., maltitol, erythritol, sorbitol), artificial sweeteners (e.g., sucralose, acesulfame potassium, stevia) and combinations thereof. A particularly desirable carbohydrate is a low dextrose equivalent (DE) maltodextrin.

### Other Optional Ingredients

The nutritional compositions as used in the methods of the present disclosure may further comprise other optional components that may modify the physical, chemical, aesthetic or processing characteristics of the products or serve as pharmaceutical or additional nutritional components when used in the targeted population. Many such optional ingredients are known or otherwise suitable for use in medical food or other nutritional products or pharmaceutical dosage forms and may also be used in the compositions herein, provided that such optional ingredients are safe for oral administration and are compatible with the essential and other ingredients in the selected product form.

Non-limiting examples of such optional ingredients include preservatives, emulsifying agents, buffers, fructooligosaccharides, galactooligosaccharides, polydextrose, and other prebiotics, probiotics, pharmaceutical actives, anti-inflammatory agents, additional nutrients as described herein, colorants, flavors, thickening agents and stabilizers, emulsifying agents, lubricants, and so forth.

The nutritional compositions may further comprise a sweetening agent, preferably including at least one sugar alcohol such as maltitol, erythritol, sorbitol, xylitol, mannitol, isolmalt, and lactitol, and also preferably including at least one artificial or high potency sweetener such as acesulfame K, aspartame, sucralose, saccharin, stevia, and tagatose. These sweetening agents, especially as a combination of a sugar alcohol and an artificial sweetener, are especially useful in formulating liquid beverage embodiments of the present disclosure having a desirable favor profile. These sweetener combinations are especially effective in masking undesirable flavors sometimes associated with the addition of vegetable proteins to a liquid beverage. Optional sugar alcohol concentrations in the nutritional product may range from at least 0.01%, including from about 0.1% to about 10%, and also including from about 1% to about 6%, by weight of the nutritional product. Optional artificial sweetener concentrations may range from at least 0.01%, including from about 0.05% to about 5%, also including from about 0.1% to about 1.0%, by weight of the nutritional product.

A flowing agent or anti-caking agent may be included in the nutritional compositions as described herein to retard clumping or caking of the powder over time and to make a powder embodiment flow easily from its container. Any known flowing or anti-caking agents that are known or otherwise suitable for use in a nutritional powder or product form are suitable for use herein, non-limiting examples of which include tricalcium phosphate, silicates, and combinations thereof. The concentration of the flowing agent or anti-caking agent in the nutritional composition varies depending upon the product form, the other selected ingredients, the desired flow properties, and so forth, but most typically range from about 0.1% to about 4%, including from about 0.5% to about 2%, by weight of the nutritional composition.

A stabilizer may also be included in the nutritional compositions. Any stabilizer that is known or otherwise suitable for use in a nutritional composition is also suitable for use herein, some non-limiting examples of which include gums such as xanthan gum. The stabilizer may represent from about 0.1% to about 5.0%, including from about 0.5% to about 3%, including from about 0.7% to about 1.5%, by weight of the nutritional composition.

The nutritional compositions may further comprise any of a variety of other vitamins or related nutrients, non-limiting examples of which include vitamin A, vitamin D, vitamin E, vitamin K, thiamine, riboflavin, pyridoxine, vitamin B₁₂, carotenoids (e.g., beta-carotene, zeaxanthin, lutein, lycopene), niacin, folic acid, pantothenic acid, biotin, vitamin C, choline, inositol, salts and derivatives thereof, and combinations thereof.

The nutritional compositions may further comprise any of a variety of other additional minerals, non-limiting examples of which include calcium, phosphorus, magnesium, iron, zinc, manganese, copper, sodium, potassium, molybdenum, chromium, chloride, and combinations thereof.

### Methods of Manufacture

The nutritional compositions of the present disclosure may be prepared by any known or otherwise effective manufacturing technique for preparing the selected product solid or liquid form. Many such techniques are known for any given product form such as nutritional liquids or powders and can easily be applied by one of ordinary skill in the art to the nutritional compositions described herein.

The nutritional compositions of the present disclosure can therefore be prepared by any of a variety of known or otherwise effective formulation or manufacturing methods. In one suitable manufacturing process, for example, at least three separate slurries are prepared, including a protein-in-fat (PIF) slurry, a carbohydrate-mineral (CHO-MIN) slurry, and a protein-in-water (PIW) slurry. The PIF slurry is formed by heating and mixing the oil (e.g., canola oil, corn oil, etc.) and then adding an emulsifier (e.g., lecithin), fat soluble vitamins, and a portion of the total protein (e.g., milk protein concentrate, etc.) with continued heat and agitation. The CHO-MIN slurry is formed by adding with heated agitation to water: minerals (e.g., potassium citrate, dipotassium phosphate, sodium citrate, etc.), trace and ultra trace minerals (TM/UTM premix), thickening or suspending agents (e.g. avicel, gellan, carrageenan). The resulting CHO-MIN slurry is held for 10 minutes with continued heat and agitation before adding additional minerals (e.g., potassium chloride, magnesium carbonate, potassium iodide, etc.), and/or carbohydrates (e.g., HMOs, fructooligosaccharide, sucrose, corn syrup, etc.). The PIW slurry is then formed by mixing with heat and agitation the remaining protein, if any.

The resulting slurries are then blended together with heated agitation and the pH adjusted to 6.6-7.0, after which the composition is subjected to high-temperature short-time (HTST) processing during which the composition is heat treated, emulsified and homogenized, and then allowed to cool. Water soluble vitamins and ascorbic acid are added, the pH is adjusted to the desired range if necessary, flavors are added, and water is added to achieve the desired total solid level. The composition is then aseptically packaged to form an aseptically packaged nutritional emulsion. This emulsion can then be further diluted, heat-treated, and packaged to form a ready-to-feed or concentrated liquid, or it can be heat-treated and subsequently processed and packaged as a reconstitutable powder, e.g., spray dried, drymixed, agglomerated.

The nutritional solid, such as a spray dried nutritional powder or drymixed nutritional powder, may be prepared by any collection of known or otherwise effective technique, suitable for making and formulating a nutritional powder.

For example, when the nutritional powder is a spray dried nutritional powder, the spray drying step may likewise include any spray drying technique that is known for or otherwise suitable for use in the production of nutritional powders. Many different spray drying methods and techniques are known for use in the nutrition field, all of which are suitable for use in the manufacture of the spray dried nutritional powders herein.

One method of preparing the spray dried nutritional powder comprises forming and homogenizing an aqueous slurry or liquid comprising predigested fat, and optionally protein, carbohydrate, and other sources of fat, and then spray drying the slurry or liquid to produce a spray dried nutritional powder. The method may further comprise the step of spray drying, drymixing, or otherwise adding additional nutritional ingredients, including any one or more of the ingredients described herein, to the spray dried nutritional powder.

Other suitable methods for making nutritional products are described, for example, in U.S. Pat. No. 6,365,218 (Borschel, et al.), U.S. Pat. No. 6,589,576 (Borschel, et al.), U.S. Pat. No. 6,306,908 (Carlson, et al.), U.S. Patent Application No. 20030118703 A1 (Nguyen, et al.), which descriptions are incorporated herein by reference to the extent that they are consistent herewith.

The present application encompasses a method of enhancing cell-mediated immunity in an individual in need thereof, the method comprising administering to the individual in need thereof a nutritional composition comprising a sialylated human milk oligosaccharide in an amount sufficient to enhance T-cell mediated responses.

The present application encompasses the method previously described wherein the individual in need thereof is selected from the group consisting of allergy-prone infants, pediatrics, teens, or adults, autoimmunity disease-prone infants, pediatrics, teens, or adults, non-breast fed infants, breastfed infants supplemented with formula or human milk fortifier, immunosenescent adults and older adults, menopausal women, post-menopausal women, chemotherapy patients, individuals with compromised immune systems, individuals with AIDS, and combinations thereof.

The present application encompasses the method previously described wherein the nutritional composition is a liquid and comprises from 0.001 mg/mL to about 20 mg/mL of sialylated human milk oligosaccharide. The present application encompasses the method previously described wherein the nutritional composition is a liquid and comprises from 0.001 mg/mL to about 10 mg/mL of sialylated human milk oligosaccharide. The present application encompasses the method previously described wherein the nutritional composition is a liquid and comprises from 0.001 mg/mL to about 5 mg/mL of sialylated human milk oligosaccharide.

The present application encompasses the method previously described wherein the nutritional composition is a powder and comprises from about 0.0005% to about 5% of sialylated human milk oligosaccharide by weight of the powder. The present application encompasses the method previously described wherein the nutritional composition is a powder and comprises from about 0.01% to about 1% of sialylated human milk oligosaccharide by weight of the powder.

The present application encompasses the method previously described wherein the sialylated human milk oligosaccharide is selected from the group consisting of 3′-Sialyllactose, 6′-Sialyllactose, lactosialotetraose, and combinations thereof.

The present application encompasses the method previously described wherein the nutritional composition is substantially free of a fucosylated human milk oligosaccharide.

The present application encompasses a method of enhancing cell-mediated immunity in an individual in need thereof, the method comprising administering to the individual in need thereof a nutritional composition comprising a fucosylated human milk oligosaccharide in an amount sufficient to enhance T-cell regulatory responses.

The present application encompasses the method previously described wherein the individual in need thereof is selected from the group consisting of allergy-prone infants, pediatrics, teens, or adults, autoimmunity disease-prone infants, pediatrics, teens, or adults, non-breast fed infants, breastfed infants supplemented with formula or human milk fortifier, immunosenescent adults and older adults, menopausal women, post-menopausal women, individuals afflicted with a chronic inflammatory disease, and combinations thereof.

The present application encompasses the method previously described wherein the nutritional composition is a liquid and comprises from 0.001 mg/mL to about 20 mg/mL of fucosylated human milk oligosaccharide. The present application encompasses the method previously described wherein the nutritional composition is a liquid and comprises from 0.001 mg/mL to about 10 mg/mL of fucosylated human milk oligosaccharide. The present application encompasses the method previously described wherein the nutritional composition is a liquid and comprises from 0.001 mg/mL to about 5 mg/mL of fucosylated human milk oligosaccharide.

The present application encompasses the method previously described wherein the nutritional composition is a powder and comprises from about 0.0005% to about 5% of fucosylated human milk oligosaccharide by weight of the powder.

The present application encompasses the method previously described wherein the nutritional composition is a powder and comprises from about 0.01% to about 1% of fucosylated human milk oligosaccharide by weight of the powder.

The present application encompasses the method previously described wherein the fucosylated human milk oligosaccharide is selected from the group consisting of 2′-Fucosyllactose, 3′-Fucosyllactose, lacto-N-fucopentaose, monofucosyllacto-N-hexaose, and combinations thereof.

The present application encompasses the method previously described wherein the nutritional composition is substantially free of a sialylated human milk oligosaccharide.

The present application encompasses a method of enhancing cell-mediated immunity in an individual in need thereof, the method comprising administering to the individual in need thereof a nutritional composition comprising a sialylated human milk oligosaccharide in an amount sufficient to enhance T-cell mediated responses and a fucosylated human milk oligosaccharide in an amount sufficient to enhance T-cell regulatory responses.

The present application encompasses the method previously described wherein the individual in need thereof is selected from the group consisting of allergy-prone infants, pediatrics, teens, or adults, autoimmunity disease-prone infants, pediatrics, teens, or adults, non-breast fed infants, breastfed infants supplemented with formula or human milk fortifier, immunosenescent adults and older adults, menopausal women, post-menopausal women, chemotherapy patients, individuals afflicted with a chronic inflammatory disease, individuals with compromised immune systems, individuals with AIDS, and combinations thereof.

The present application encompasses the method previously described wherein the nutritional composition is a liquid and comprises from 0.001 mg/mL to about 20 mg/mL of sialylated and fucosylated human milk oligosaccharide. The present application encompasses the method previously described wherein the nutritional composition is a liquid and comprises from 0.001 mg/mL to about 10 mg/mL of sialylated and fucosylated human milk oligosaccharide. The present application encompasses the method previously described wherein the nutritional composition is a liquid and comprises from 0.001 mg/mL to about 5 mg/mL of sialylated and fucosylated human milk oligosaccharide.

The present application encompasses the method previously described wherein the nutritional composition is a powder and comprises from about 0.0005% to about 5% of sialylated and fucosylated human milk oligosaccharide by weight of the powder. The present application encompasses the method previously described wherein the nutritional composition is a powder and comprises from about 0.01% to about 1% of sialylated and fucosylated human milk oligosaccharide by weight of the powder.

The present application encompasses the method previously described wherein the sialylated human milk oligosaccharide is selected from the group consisting of 3′-sialyllactose, 6′-sialyllactose, lactosialotetraose, and combinations thereof and the fucosylated human milk oligosaccharide is selected from the group consisting of 2′-fucosyllactose, 3′-fucosyllactose, lacto-N-fucopentaose, monofucosyllacto-N-hexaose, and combinations thereof.

The present application encompasses a method of enhancing T-cell mediated responses in an individual in need thereof, the method comprising administering to the individual in need thereof a nutritional composition comprising a sialylated human milk oligosaccharide in an amount sufficient to enhance T-cell mediated responses.

The present application encompasses a method of enhancing T-cell regulatory responses in an individual in need thereof, the method comprising administering to the individual in need thereof a nutritional composition comprising a fucosylated human milk oligosaccharide in an amount sufficient to enhance T-cell regulatory responses.

All percentages, parts and ratios as used herein, are by weight of the total composition, unless otherwise specified. All such weights, as they pertain to listed ingredients, are based on the active level and, therefore, do not include solvents or by-products that may be included in commercially available materials, unless otherwise specified.

Numerical ranges as used herein are intended to include every number and subset of numbers within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The various embodiments of the nutritional compositions of the present disclosure may also be substantially free of any optional or selected ingredient or feature described herein, provided that the remaining nutritional composition still contains all of the required ingredients or features as described herein. In this context, and unless otherwise specified, the term “substantially free” means that the selected nutritional composition contains less than a functional amount of the optional ingredient, typically less than 1%, including less than 0.5%, including less than 0.1%, and also including zero percent, by weight of such optional or selected ingredient.

The nutritional compositions and methods may comprise, consist of, or consist essentially of the essential elements of the compositions and methods as described herein, as well as any additional or optional element described herein or otherwise useful in nutritional product applications.

While the present disclosure has illustrated by description several embodiments and while the illustrative embodiments have been described in considerable detail, it is not the intention of the applicant to restrict or in any way limit the scope of the appended claims to such detail. Additional advantages and modifications may readily appear to those skilled in the art.

### EXAMPLES

The following examples illustrate specific embodiments and/or features of the nutritional compositions of the present disclosure. The examples are given solely for the purpose of illustration and are not to be construed as limitations of the present disclosure, as many variations thereof are possible without departing from the spirit and scope of the disclosure. All exemplified amounts are weight percentages based upon the total weight of the composition, unless otherwise specified.

The exemplified compositions are shelf stable nutritional compositions prepared in accordance with the manufacturing methods described herein, such that each exemplified composition, unless otherwise specified, includes an aseptically processed embodiment and a retort packaged embodiment.

### Examples 1-5

Examples 1-5 illustrate ready-to-feed nutritional emulsions of the present disclosure, the ingredients of which are listed in the table below. All ingredient amounts are listed as kilogram per 1000 kilogram batch of product, unless otherwise specified.

### Examples 1-5

| Ingredient | Ex. 1 | Ex. 2 | Ex. 3 | Ex. 4 | Ex. 5 |
|---|---|---|---|---|---|
| Water | Q.S. | Q.S. | Q.S. | Q.S. | Q.S. |
| Condensed Skim Milk | 86.64 | 86.64 | 86.64 | 86.64 | 86.64 |
| Lactose | 54.80 | 54.80 | 54.80 | 54.80 | 54.80 |
| High oleic safflower oil | 14.10 | 14.10 | 14.10 | 14.10 | 14.10 |
| Soybean oil | 10.6 | 10.6 | 10.6 | 10.6 | 10.6 |
| Coconut oil | 10.1 | 10.1 | 10.1 | 10.1 | 10.1 |
| 3′ sialylallactose (3′SL) | 0.0948 | 0.090 | 0.085 | 9.479 | 9.005 |
| Galactooligosaccharides (GOS) | 8.63 | 8.63 | 8.63 | 8.63 | 8.63 |
| Whey protein concentrate | 6.40 | 6.40 | 6.40 | 6.40 | 6.40 |
| Potassium citrate | 478.9 g | 478.9 g | 478.9 g | 478.9 g | 478.9 g |
| Calcium carbonate | 448.28 g | 448.28 g | 448.28 g | 448.28 g | 448.28 g |
| Soy lecithin | 355.74 g | 355.74 g | 355.74 g | 355.74 g | 355.74 g |
| Stabilizer | 355.74 g | 355.74 g | 355.74 g | 355.74 g | 355.74 g |
| ARA oil | 368.01 g | 368.01 g | 368.01 g | 368.01 g | 368.01 g |
| Nucleotide/chloride premix | 293.26 g | 293.26 g | 293.26 g | 293.26 g | 293.26 g |
| Potassium chloride | 226.45 g | 226.45 g | 226.45 g | 226.45 g | 226.45 g |
| Ascorbic acid | 445.94 g | 445.94 g | 445.94 g | 445.94 g | 445.94 g |
| Vitamin mineral premix | 142.88 g | 142.88 g | 142.88 g | 142.88 g | 142.88 g |
| DHA oil | 137.8 g | 137.8 g | 137.8 g | 137.8 g | 137.8 g |
| Carrageenan | 180.0 g | 180.0 g | 180.0 g | 180.0 g | 180.0 g |
| Magnesium chloride | 55.0 g | 55.0 g | 55.0 g | 55.0 g | 55.0 g |
| Ferrous sulfate | 58.0 g | 58.0 g | 58.0 g | 58.0 g | 58.0 g |
| Choline chloride | 53.9 g | 53.9 g | 53.9 g | 53.9 g | 53.9 g |
| Vitamin A, D₃, E, K₁ premix | 47.4 g | 47.4 g | 47.4 g | 47.4 g | 47.4 g |
| Citric acid | 29.77 g | 29.77 g | 29.77 g | 29.77 g | 29.77 g |
| Mixed carotenoid premix | 26.40 g | 26.40 g | 26.40 g | 26.40 g | 26.40 g |
| Sodium chloride | AN | AN | AN | AN | AN |
| L-carnitine | 3.31 g | 3.31 g | 3.31 g | 3.31 g | 3.31 g |
| Tricalcium phosphate | 15.65 g | 15.65 g | 15.65 g | 15.65 g | 15.65 g |
| Potassium phosphate monobasic | 13.67 g | 13.67 g | 13.67 g | 13.67 g | 13.67 g |
| Riboflavin | 2.42 g | 2.42 g | 2.42 g | 2.42 g | 2.42 g |
| Potassium hydroxide | AN | AN | AN | AN | AN |

AN = as needed

### Examples 6-10

Examples 6-10 illustrate ready-to-feed nutritional emulsions of the present disclosure, the ingredients of which are listed in the table below. All ingredient amounts are listed as kilogram per 1000 kilogram batch of product, unless otherwise specified.

### Examples 6-10

| Ingredient | Ex. 6 | Ex. 7 | Ex. 8 | Ex. 9 | Ex. 10 |
|---|---|---|---|---|---|
| Water | Q.S. | Q.S. | Q.S. | Q.S. | Q.S. |
| Condensed Skim Milk | 86.64 | 86.64 | 86.64 | 86.64 | 86.64 |
| Lactose | 54.80 | 54.80 | 54.80 | 54.80 | 54.80 |
| High oleic safflower oil | 14.10 | 14.10 | 14.10 | 14.10 | 14.10 |
| Soybean oil | 10.6 | 10.6 | 10.6 | 10.6 | 10.6 |
| Coconut oil | 10.1 | 10.1 | 10.1 | 10.1 | 10.1 |
| HMO Mixture | 0.0948 | 0.0901 | 0.0853 | 9.479 | 9.0047 |
| 6′ sialyllactose (6′SL) | 0.0316 | 0.0300 | 0.0284 | 0 | 0 |
| 2′fucosyllactose (2′FL) | 0.0316 | 0.0300 | 0.0284 | 3.159 | 3.002 |
| Lacto-N-neotetraose (LNnT) | 0.0316 | 0.0300 | 0.0284 | 0 | 0 |
| Galactooligosaccharides (GOS) | 8.63 | 8.63 | 8.63 | 8.63 | 8.63 |
| Whey protein concentrate | 6.40 | 6.40 | 6.40 | 6.40 | 6.40 |
| Potassium citrate | 478.9 g | 478.9 g | 478.9 g | 478.9 g | 478.9 g |
| Calcium carbonate | 448.28 g | 448.28 g | 448.28 g | 448.28 g | 448.28 g |
| Soy lecithin | 355.74 g | 355.74 g | 355.74 g | 355.74 g | 355.74 g |
| Stabilizer | 355.74 g | 355.74 g | 355.74 g | 355.74 g | 355.74 g |
| ARA oil | 368.01 g | 368.01 g | 368.01 g | 368.01 g | 368.01 g |
| Nucleotide/chloride premix | 293.26 g | 293.26 g | 293.26 g | 293.26 g | 293.26 g |
| Potassium chloride | 226.45 g | 226.45 g | 226.45 g | 226.45 g | 226.45 g |
| Ascorbic acid | 445.94 g | 445.94 g | 445.94 g | 445.94 g | 445.94 g |
| Vitamin mineral premix | 142.88 g | 142.88 g | 142.88 g | 142.88 g | 142.88 g |
| DHA oil | 137.8 g | 137.8 g | 137.8 g | 137.8 g | 137.8 g |
| Carrageenan | 180.0 g | 180.0 g | 180.0 g | 180.0 g | 180.0 g |
| Magnesium chloride | 55.0 g | 55.0 g | 55.0 g | 55.0 g | 55.0 g |
| Ferrous sulfate | 58.0 g | 58.0 g | 58.0 g | 58.0 g | 58.0 g |
| Choline chloride | 53.9 g | 53.9 g | 53.9 g | 53.9 g | 53.9 g |
| Vitamin A, D3, E, K1 premix | 47.40 g | 47.40 g | 47.40 g | 47.40 g | 47.40 g |
| Citric acid | 29.77 g | 29.77 g | 29.77 g | 29.77 g | 29.77 g |
| Mixed carotenoid premix | 26.40 g | 26.40 g | 26.40 g | 26.40 g | 26.40 g |
| Sodium chloride | AN | AN | AN | AN | AN |
| L-carnitine | 3.31 g | 3.31 g | 3.31 g | 3.31 g | 3.31 g |
| Tricalcium phosphate | 15.65 g | 15.65 g | 15.65 g | 15.65 g | 15.65 g |
| Potassium phosphate monobasic | 13.67 g | 13.67 g | 13.67 g | 13.67 g | 13.67 g |
| Riboflavin | 2.42 g | 2.42 g | 2.42 g | 2.42 g | 2.42 g |
| Potassium hydroxide | AN | AN | AN | AN | AN |

AN = as needed

### Example 11

In this Example, the effects of human milk oligosaccharides (HMOs) on T-cell mediated immune responses were analyzed.

The HMOs or HMO mixtures used in this Example included 2′-fucosyllactose (2′FL), 3-fucosyllactose (3′FL), 3′-sialyllactose (3′SL), 6′-sialyllactose (6′SL), Lacto-N-neotetraose (LNnT), a FL mixture (FL mix) containing 2′FL and 3FL at a ratio of 2′FL to 3FL of 85:15, a SL mixture (SL mix) containing sialic acid, 3′SL, and 6′SL in a ratio of sialic acid to 3′SL to 6′SL of 50:10:40, and a mixture of HMOs (HMO mix) isolated from human milk. The composition of the HMO mixture is shown in the table below. Compositions were determined by HPLC-chip TOF MS.

| | | |
|---|---|---|
| Classification | HMO | % of total |

| | | | |
|---|---|---|---|
| Non-fucosylated, non- | LNT/LNnT | 16.1 | 56.1 |
| sialylated | | | |
| Fucosylated | LNFP | 8.9 | |
| | MFLNH | 6.7 | |
| | 2′FL | 5.8 | |
| Sialylated | LST | 11.1 | 31.6 |
| | 6′SL | 2.1 | |
| | 3′SL | 1.1 | |
| Fucosylated & | FS-LNnH | 4.4 | 12.4 |
| Sialylated | FS-LNH | 3.1 | |
| | F-LST | 2.3 | |

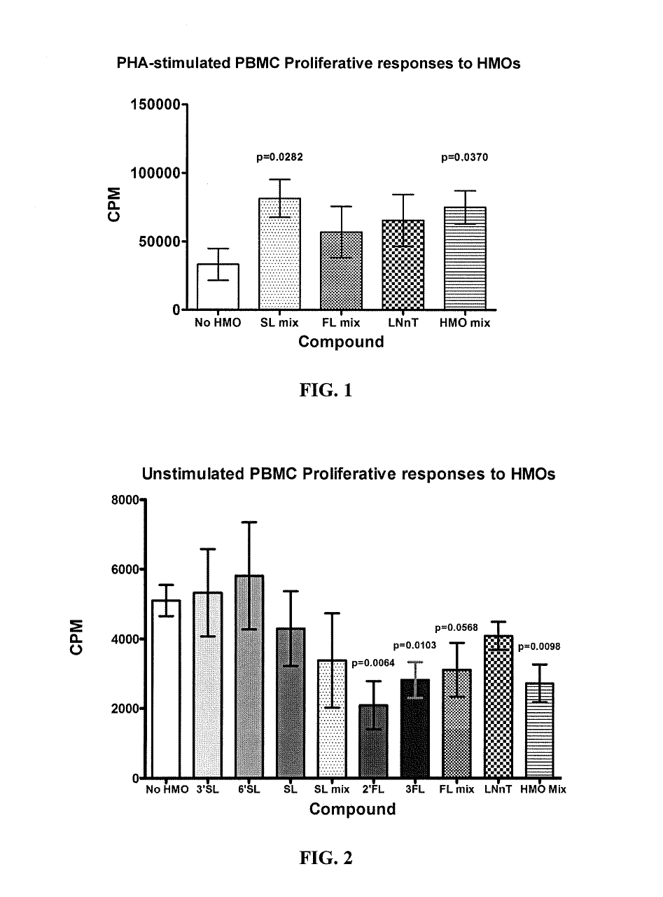

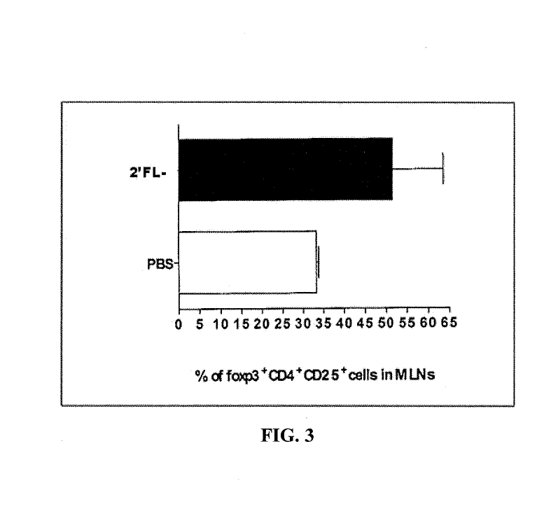

T-cell mediated responses were assessed by proliferative responses to the T-cell mitogen phytohemagglutinin (PHA) (simulates infection). Blood was collected into heparinized Vacutainer™ tubes from five, ten-day old piglets that had been fed an artificial sow formula from birth; this artificial sow formula did not contain any human milk oligosaccharides. Peripheral blood mononuclear cells (PBMCs) were isolated using standard density gradient (Ficoll-Paque PLUS) techniques. Residual red blood cells were lysed with a hypotonic ammonium chloride solution. Cells were washed and resuspended in RPMI (Roswell Park Memorial Institute medium) media containing: 10% fetal bovine serum, 2 mM Glutamine, 1 mM sodium pyruvate, 20 mM HEPES (4-(2-hydroxyethyl)-1-piperazineethanesulfonic acid), 50 μg/mL gentamicin, 1000 IU/mL penicillin and 100 μg/mL streptomycin, at a concentration of 1×10⁶/mL, and then cultured in the presence or absence of the individual HMOs or HMO mixtures at a concentration of 125 μg/mL, alone or in combination with 25 μg/mL PHA. Cells were cultured for 72 hours, and cellular proliferation was determined using standard tritiated thymidine incorporation methodology. The results are shown in FIGS. 1 and 2.

As shown in FIG. 1, the addition of sialyllactose (SL), through both the SL mix and the HMO mix, to cell cultures in the presence of PHA enhanced peripheral blood mononuclear cell proliferation. This is an unexpected finding as immune modification by HMOs has previously been associated with anti-inflammatory effects or modulation of the innate immune system (Jantscher-Krenn E, et al, “Human Milk Oligosaccharides and Their Potential Benefits for the Breast-Fed Neonate”, Minerva Pediatr 64: 83-99, 2012). Furthermore, Kuntz et al (2008) reported that SL exposure to gastrointestinal epithelial cells decreases proliferation and increases differentiation (Kuntz S, et al, “Oligosaccharides from Human Milk Influence growth-related Characteristics of Intestinally Transformed and Non-Transformed Intestinal Cells”, Br J of Nutr, 99: 462-471, 2008). Consistent with the Kuntz report, Eiwegger et al. (2004) reported modulation of cord blood T-cell induction of some cytokines associated with differentiation from naïve to type 1 (IFN-γ) and increased percentages of CD4+ T-cells co-expressing CD25 (an activation marker) in the presence of a combination of eight acidic oligosaccharides: 3′SL, 6′SL, sialic acid, fucosy-sialyl-lacto-N-neohexaose, Lactosialyl-tetrasaccharide (LST)a, LSTb, LSTc, and disialyl-lacto N-tetraose (DSLNT) (Eiwegger T, et al., “Human Milk-Derived Oligosaccharides and Plant-Derived Oligosaccharides Stimulate Cytokine Production of Cord Blood T-Cells in Vitro”, Pediatr Res 56: 536-540, 2004).

Fucosyllactose (FL) did not significantly enhance proliferation nor did LNnT, however, the HMO mix, which contained SL, FL, and LNnT, enhanced proliferation compared to cultures containing no HMO. Taken together, these findings suggest that 3′SL and/or 6′SL may be the key component(s) in the HMO mix that drove significantly enhanced peripheral blood mononuclear cell proliferation. Although the SL mix contained free sialic acid, there was no free sialic acid in the HMO mix indicating that enhanced proliferation occurred without the presence of free sialic acid.

As shown in FIG. 2, the addition of FL to cell cultures decreased unstimulated peripheral blood mononuclear cell proliferation. This observation was noted in cell cultures containing 2′FL, 3′FL, the FL mix, and the HMO mix but this was not seen in cultures containing any forms of SL or LNnT, indicating the component in the HMO mix driving the observation may be FL.

These data demonstrate the differential effects of SL and FL on T-cell mediated immunity. Where SL increased PBMC proliferation in PHA stimulated cells, FL decreased PBMC proliferation in unstimulated cells. Furthermore, these compounds influence proliferation in a manner that is not mutually exclusive, i.e., the HMO mix contained both SL and FL compounds, yet neither form of HMO inhibited the attributes of the other. The HMO mix increased proliferation in PHA-stimulated cell cultures similar to the SL mix while the HMO mix decreased proliferation in unstimulated cell cultures similar to the FL compounds.

The modulation of cell-mediated immunity is beneficial as in vitro PHA-stimulation is generally recognized as a surrogate for in vivo cell-mediated immunity to infectious microbial antigens or altered “self”. The increase in PHA proliferative response by forms of SL indicated enhancement of cell-mediated immunity, and thereby, increased resistance to intracellular microbial pathogens, fungi, protozoa and tumor cells.

Further, after the induction of a cellular response and the attenuation of the threat, the body must rid itself of the accumulation of an amplified number of effector cells, which are now quiescent or unstimulated. Because forms of FL decreased proliferation of non-activated cells, FL may enhance resolution of the immune response and thereby attenuate the development of an uncontrolled inflammatory response. In combination, as indicated by the HMO mix, SL and FL increased resistance to intracellular microbial pathogens (virus and bacteria) fungi, protozoa, and tumor cells, while enhancing resolution of the immune response thereby preventing uncontrolled or chronic inflammatory response.

### Example 12

In this Example, the effects of 2′FL on T-cell regulation were analyzed and evaluated.

Mice were orally supplemented with 7.5 mg of 2′FL in 200 μL of PBS (n=5) or 200 μL of PBS (n=5) daily for 5 days. On day six, mice were sacrificed and lymphocytes from mesenteric lymph nodes were removed and isolated using standard methods. Mesenteric lymph node lymphocytes (MLNLs) were stained with fluorochrome labeled monoclonal antibodies specific for CD4 and CD25 surface markers. Cells were then fixed with a paraformaldehyde solution to maintain surface marker staining integrity. Stained MLNLs were permeabilized with a saponin-based solution to allow for intracellular uptake of monoclonal antibodies and then stained with a fluorochrome labeled monoclonal antibody specific for the transcription factor FoxP3. All cell preparation methods used were based on recommendations of the monoclonal antibody manufacturer. MLNL suspensions were analyzed by flow cytometery. FoxP3⁺, CD4⁺, and CD25⁺ cells are classified T regulatory cells.

T-regulatory cells that co-express FoxP3, CD4, and CD25 are thymically derived. These cells play a critical role in preventing aberrant responses to self and non-self antigens resulting in, for example, prevention of autoimmune diseases and allergic diseases. Thus, 2′FL modulated cell-mediated immunity by prevention of aberrant immune responses by the generation of T regulatory cells (FIG. 3) as well as by resolution or maintenance of appropriate immune responses by dampening proliferation of non-activated cells as shown in Example 11 (FIG. 2).

In summary, the data of Examples 11 and 12 support the development/enhancement of appropriately regulated T-cell-mediated immune responses by a combination of SL and FL. The benefit of this combination of oligosaccharides is increased resistance to intracellular microbial pathogens, fungi, protozoa, and tumor cells, increased resolution of infectious and/or inflammatory processes (chronic inflammation) and prevention of autoimmune and allergic diseases. SL alone may be used to enhance deficient T-cell-mediated immune responses in those that are in need of T-cell immune enhancement, including but not limited to, the elderly, or those recovering from forms of immune suppressing therapies such as chemotherapy, or those afflicted with an immune suppressing condition such as HIV. FL alone may be used to prevent or treat over-exuberant immune responses, including but not limited to, allergic, autoimmune diseases, and chronic inflammatory diseases.

What is claimed is:

1. A method of enhancing cell-mediated immunity in an individual in need thereof, the method comprising administering to the individual in need thereof a nutritional composition comprising at least one of a sialylated human milk oligosaccharide and a fucosylated human milk oligosaccharide in an amount sufficient to enhance cell-mediated immunity.

2. The method of claim 1, wherein the individual in need thereof is selected from the group consisting of allergy-prone infants, pediatrics, teens, or adults; autoimmunity disease-prone infants, pediatrics, teens, or adults; non-breast fed infants; breastfed infants supplemented with formula or human milk fortifier; immunosenescent adults and older adults; menopausal women; post-menopausal women; chemotherapy patients; individuals with compromised immune systems; individuals with AIDS; and combinations thereof.

3. The method of claim 1, wherein the nutritional composition is a liquid comprising from about 0.001 mg/mL to about 20 mg/mL of either the sialylated human milk oligosaccharide or the fucosylated human milk oligosaccharide.

4. The method of claim 1, wherein the nutritional composition is a powder comprising from about 0.0005% to about 5% of either the sialylated human milk oligosaccharide or the fucosylated human milk oligosaccharide, by weight of the powder.

5. The method of claim 1, wherein the sialylated human milk oligosaccharide is selected from 3′-sialyllactose, 6′-sialyllactose, lactosialotetraose, and combinations thereof.

6. The method of claim 1, wherein the nutritional composition is substantially free of fucosylated human milk oligosaccharides.

7. The method of claim 1, wherein the fucosylated human milk oligosaccharide is selected from 2′-fucosyllactose, 3′-fucosyllactose, lacto-N-fucopentaose, monofucosyllacto-N-hexaose, and combinations thereof.

8. The method of claim 1, wherein the nutritional composition is substantially free of sialylated human milk oligosaccharides.

9. The method of claim 1, wherein the nutritional composition comprises the sialylated human milk oligosaccharide in an amount sufficient to enhance T-cell mediated responses and the fucosylated human milk oligosaccharide in an amount sufficient to enhance T-cell regulatory responses.

10. The method of claim 1, wherein the nutritional composition is a liquid comprising from about 0.001 mg/mL to about 20 mg/mL of each of the sialylated and the fucosylated human milk oligosaccharides.

11. The method of claim 1, wherein the nutritional composition is a powder comprising from about 0.0005% to about 5% of each of the sialylated and the fucosylated human milk oligosaccharides, by weight of the powder.

12. The method of claim 1, wherein the sialylated human milk oligosaccharide is selected from 3′-sialyllactose, 6′-sialyllactose, lactosialotetraose, and combinations thereof and the fucosylated human milk oligosaccharide is selected from 2′-fucosyllactose, 3′-fucosyllactose, lacto-N-fucopentaose, monofucosyllacto-N-hexaose, and combinations thereof.

13. The method of claim 1, wherein the nutritional composition comprises the sialylated human milk oligosaccharide in an amount sufficient to enhance T-cell mediated responses.

14. The method of claim 1, wherein the nutritional composition comprises the fucosylated human milk oligosaccharide in an amount sufficient to enhance T-cell regulatory responses.

15. The method of claim 3, wherein the nutritional composition comprises from about 0.001 mg/mL to about 10 mg/mL of either the sialylated human milk oligosaccharide or the fucosylated human milk oligosaccharide.

16. The method of claim 3, wherein the nutritional composition comprises from about 0.001 mg/mL to about 5 mg/mL of either the sialylated human milk oligosaccharide or the fucosylated human milk oligosaccharide.

17. The method of claim 4, wherein the nutritional composition comprises from about 0.01% to about 1% of either the sialylated human milk oligosaccharide or the fucosylated human milk oligosaccharide, by weight of the powder.

18. The method of claim 10, wherein the nutritional composition comprises from about 0.001 mg/mL to about 10 mg/mL of each of the sialylated human milk oligosaccharide and the fucosylated human milk oligosaccharide.

19. The method of claim 10, wherein the nutritional composition comprises from about 0.001 mg/mL to about 5 mg/mL of each of the sialylated human milk oligosaccharide and the fucosylated human milk oligosaccharide.

20. The method of claim 11, wherein the nutritional composition comprises from about 0.01% to about 1% of each of the sialylated human milk oligosaccharide and the fucosylated human milk oligosaccharide, by weight of the powder.
